(19) United States
(12) Patent Application Publication
(10) Pub. No.: US 2025/0302908 A1
(43) Pub. Date: Oct. 2, 2025

(54) COMPOSITION, KIT, AND METHOD FOR TREATMENT OF NOSEBLEEDS

(71) Applicant: Ask Moms, LLC, Clarkston, MI (US)

(72) Inventors: Amy Duncan, Clarkston, MI (US); Kathleen Kuron, Clarkston, MI (US); Nicole Cadena, Clarkston, MI (US)

(21) Appl. No.: 19/093,951

(22) Filed: Mar. 28, 2025

Related U.S. Application Data
(60) Provisional application No. 63/572,513, filed on Apr. 1, 2024.

(51) Int. Cl. A61K 38/01; A61K 9/00; A61K 9/14; A61K 47/46

(52) U.S. Cl. CPC A61K 38/014; A61K 9/006; A61K 9/14; A61K 47/46

(57) ABSTRACT

A composition for treatment of nosebleeds includes gelatin in a range of about 50% to about 67% by volume in powdered form, flavoring in a range of about 23% to about 37% by volume in powdered form, and sweetener in a range of about 5% to about 20% by volume in powdered form.

### CROSS-REFERENCE TO RELATED APPLICATIONS

This application claims priority to and benefit of U.S. Provisional Application No. 63/572,513, filed on Apr. 1, 2024, which is incorporated herein by reference in its entirety.

### TECHNICAL FIELD

The present invention relates to an apparatus and composition for cessation of a nosebleed and a method of treatment for a nosebleed. More specifically the present invention relates to an apparatus and composition that are applied under the upper lip of a person to stop a nosebleed.

### BACKGROUND

Nosebleeds are a frequent and messy problem for many people, especially children. Known approaches for stopping a nosebleed are not reliable and can take several minutes to work, if at all. For example, a common method for stopping a person's nosebleed includes applying a tissue or other absorptive material to or within the nostrils of the person. The absorptive material soaks up the blood, but this approach is not reliable for stopping the nosebleed and may take an extended period of time to work during which the person has to keep the absorptive material applied.

A need therefore exists for a reliable and quick method of treatment to stop a nosebleed. It would be beneficial if the method utilized just a few components and could be executed in just a few steps. It would be further beneficial if the components of the method were available in a kit and were kept sterile until use.

### SUMMARY

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

According to certain aspects of the present disclosure, a composition for treatment of nosebleeds includes gelatin in a range of about 50% to about 67% by volume in powdered form, flavoring in a range of about 23% to about 37% by volume in powdered form, and sweetener in a range of about 5% to about 20% by volume in powdered form.

According to some features of the above aspects, the composition includes powdered gelatin in a range of about 50% to about 67% by volume, powdered flavoring in a range of about 23% to about 37% by volume, and powdered sweetener in a range of about 5% to about 20% by volume.

According to some features of the above aspects, the composition includes powdered gelatin in a range of about 54% to about 64% by volume, powdered flavoring in a range of about 25% to about 33% by volume, and powdered sweetener in a range of about 5% to about 18% by volume.

According to some features of the above aspects, the powdered sweetener includes one or both of sugar and monk fruit sweetener.

According to some features of the above aspects, the powdered flavoring includes powdered dried fruit.

According to some features of the above aspects, the powdered dried fruit includes powdered dried strawberries or powdered dried cherries.

According to some features of the above aspects, the composition further includes powdered shelf stabilizer in a range of about 2% to about 6% by volume.

According to some features of the above aspects, the powdered shelf stabilizer includes arrowroot powder.

According to some features of the above aspects, a kit includes a first package containing the composition as noted above, and a second package containing at least one applicator for application of the composition.

According to some features of the above aspects, the kit further includes an outer package containing the first package and the second package.

According to some features of the above aspects, the second package contains two or more of the applicators.

According to some features of the above aspects, the first package and the second package are sterile packages.

According to some features of the above aspects, the first sterile package contains at least about 1/12^{th} of a teaspoon of the composition.

According to some features of the above aspects, one or more of the gelatin, the flavoring, and the sweetener is in a gel or cream form.

According to certain aspects of the present disclosure, a method of treatment for a nosebleed in a patient includes the steps of providing a composition, and applying an effective amount of the composition under the patient's upper lip. The composition includes powdered gelatin in a range of about 50% to about 67% by volume, powdered flavoring in a range of about 23% to about 37% by volume, and powdered sweetener in a range of about 5% to about 20% by volume.

According to some features of the above aspects, the applying further includes the steps of providing an applicator, pressing the tip of the applicator into the composition to pick up the composition, and distributing the composition under the upper lip of the patient on either side of the labial frenulum via the tip of the applicator.

According to some features of the above aspects, the distributing further includes distributing the composition under the upper lip of the patient on either side of the labial frenulum via the tip of the applicator for a period of time between about a minute and about two minutes.

According to some features of the above aspects, the method further includes, before pressing the tip of the applicator into the composition to pick up the composition, wetting the tip of the applicator.

According to some features of the above aspects, the effective amount of the composition is about 1/12^{th} of a teaspoon of the composition.

According to some features of the above aspects, the method further includes after the distributing, waiting about two minutes to determine if the nosebleed has stopped. Responsive to the nosebleed continuing, the method further includes pressing the tip of the applicator or a tip of a second applicator into the composition to pick up the composition, and distributing the composition under the upper lip of the patient on either side of the labial frenulum via the tip of the applicator or the tip of the second applicator.

### BRIEF DESCRIPTION OF THE DRAWINGS

For a more complete understanding, reference is now made to the following description taken in conjunction with the accompanying drawings in which:

FIG. 1 shows a schematic representation of an exemplary composition, according to certain aspects of the present disclosure.

FIG. 2 shows a schematic representation of another exemplary composition, according to certain aspects of the present disclosure.

FIG. 3 shows a schematic drawing of an exemplary applicator, according to certain aspects of the present disclosure.

FIG. 4 shows a flow diagram of an exemplary method of treatment for a nosebleed, according to certain aspects of the present disclosure.

FIG. 5 shows a schematic drawing of a person's mouth illustrating application of the method of treatment of FIG. 4, according to certain aspects of the present disclosure.

FIG. 6 shows a schematic drawing of an exemplary kit containing a composition and an applicator, according to certain aspects of the present disclosure.

FIG. 7 shows a schematic drawing of another exemplary kit containing a composition and an applicator, according to certain aspects of the present disclosure.

### DETAILED DESCRIPTION

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

As described in detail herein an effective amount of a composition 100 applied under the upper lip near the labial frenulum where the upper lip meets the gums of a person suffering from a nosebleed has been found to be effective in stopping the nosebleed. In the description that follows, the composition 100 is intended to be descriptive of several different embodiments having constituent mixtures that vary slightly. In an embodiment the composition 100 is in a powdered form.

In other embodiments, one or more of the constituents that make up the composition 100 are in gel or cream form. For those embodiments having one or more or the constituents in gel or cream form, the one or more of the constituents is mixed with a liquid, for example, water, or any liquid suitable for rendering a powder into a gel or a cream. The composition 100 comprised of one or more constituents so mixed with the liquid is packaged for use as at least a partial gel or a partial cream instead of as a powder. Regardless of whether the packaged form of the composition 100 includes a gel or a cream, the underlying powder that makes up the composition 100 has the same volumetric proportions as described herein for composition 100 packaged as a powder.

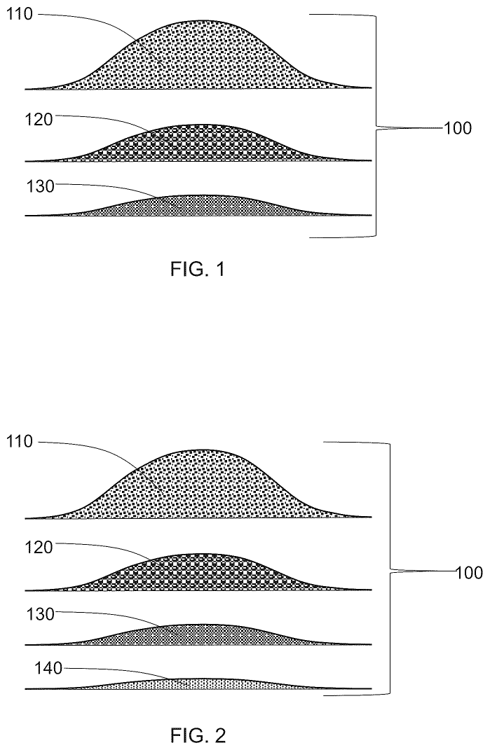

Referring to FIG. 1, in an embodiment, the composition 100 comprises gelatin 110, flavoring 120, and sweetener 130. In an embodiment, the gelatin 110, the flavoring 120, and the sweetener 130 are all powdered. In other embodiments, one or more of the gelatin 110, flavoring 120, and sweetener 130 is in a gel or cream form.

In an embodiment the composition 100 in a powdered form comprises powdered gelatin 110, powdered flavoring 120, and powdered sweetener 130. In terms of volumetric proportions of the constituents, in an embodiment of the composition 100, the powdered gelatin 100 makes up about 16 parts of the composition 100 by volume, the powdered flavoring 120 makes up about 8 parts of the composition 100 by volume, and the powdered sweetener 130 makes up about 2 parts of the composition 100 by volume. In terms of percentages of the constituents, in an embodiment where the powdered sweetener 130 makes up about 2 parts of the composition 100 by volume, the powdered gelatin 110 makes up a range of about 55% to about 67% of the composition 100 by volume, the powdered flavoring 120 makes up a range of about 25% to about 37% of the composition 100 by volume, and the powdered sweetener 130 makes up a range of about 5% to about 10% of the composition 100 by volume. In another embodiment where the powdered sweetener 130 makes up about 2 parts of the composition 100 by volume, the powdered gelatin 110 makes up a range of about 58% to about 64% of the composition 100 by volume, the powdered flavoring 120 makes up a range of about 28% to about 33% of the composition 100 by volume, and the powdered sweetener 130 makes up a range of about 5% to about 10% of the composition 100 by volume. In a further embodiment where the powdered sweetener 130 makes up about 2 parts of the composition 100 by volume, the powdered gelatin 110 makes up about 61.5% of the composition 100 by volume, the powdered flavoring 120 makes up about 30.8% of the composition 100 by volume, and the powdered sweetener 130 makes up about 7.7% of the composition 100 by volume.

In another embodiment of the composition 100, in terms of volumetric proportions of the constituents, the powdered gelatin 110 makes up about 16 parts of the composition 100 by volume, the powdered flavoring 120 makes up about 8 parts of the composition 100 by volume, and the powdered sweetener 130 makes up about 4 parts of the composition 100 by volume. In terms of percentages of the constituents, in an embodiment where the powdered sweetener 130 makes up about 4 parts of the composition 100 by volume, the powdered gelatin 110 makes up a range of about 50% to about 64% of the composition 100 by volume, the powdered flavoring 120 makes up a range of about 23% to about 35% of the composition 100 by volume, and the powdered sweetener 130 makes up a range of about 10% to about 20% of the composition 100 by volume. In another embodiment where the powdered sweetener 130 makes up about 4 parts of the composition 100 by volume, the powdered gelatin 110 makes up a range of about 54% to about 61% of the composition 100 by volume, the powdered flavoring 120 makes up a range of about 25% to about 32% of the composition 100 by volume, and the powdered sweetener 130 makes up a range of about 12% to about 18% of the composition 100 by volume. In a further embodiment where the powdered sweetener 130 makes up about 4 parts of the composition 100 by volume, the powdered gelatin 110 makes up about 57.1% of the composition 100 by volume, the powdered flavoring 120 makes up about 28.6% of the composition 100 by volume, and the powdered sweetener 130 makes up a range of about 14.3% of the composition 100 by volume.

In yet another embodiment of the composition 100, the powdered gelatin 110 makes up about 16 parts of the composition 100 by volume, the powdered flavoring 120 makes up about 8 parts of the composition 100 by volume, and the powdered sweetener 130 makes up a range of about 2 parts to about 4 parts of the composition 100 by volume. In an embodiment where the powdered sweetener 130 makes up a range of about 2 parts to about 4 parts of the composition 100 by volume, in terms of percentages of the constituents, the powdered gelatin 110 makes up a range of about 50% to about 67% of the composition 100 by volume, the powdered flavoring 120 makes up a range of about 23% to about 36% of the composition 100 by volume, and the powdered sweetener 130 makes up a range of about 5% to about 18% of the composition 100 by volume. In another embodiment where the powdered sweetener 130 makes up about 2 parts to about 4 parts of the composition 100 by volume, in terms of percentages of the constituents, the powdered gelatin 110 makes up a range of about 55% to about 63% of the composition 100 by volume, the powdered flavoring 120 makes up a range of about 26% to about 33% of the composition 100 by volume, and the powdered sweetener 130 makes up a range of about 6% to about 17% of the composition 100 by volume.

Referring to FIG. 2, in another embodiment, the composition 100 comprises the gelatin 110, the flavoring 120, the sweetener 130, and also includes a shelf stabilizer 140. In an embodiment, the gelatin 110, the flavoring 120, the sweetener 130, and the shelf stabilizer 140 are all powdered. In other embodiments, one or more of the gelatin 110, flavoring 120, sweetener 130, and shelf stabilizer 140 is in a gel or cream form. In terms of volumetric proportions, in an embodiment of the composition 100, the powdered gelatin 110 makes up about 16 parts of the composition 100 by volume, the powdered flavoring 120 makes up about 8 parts of the composition 100 by volume, the powdered sweetener 130 makes up about 2 parts of the composition 100 by volume, and the powdered shelf stabilizer 140 makes up about 1 part of the composition 100 by volume. In an embodiment where the powdered shelf stabilizer 140 makes up about 1 part of the composition 100 by volume, in terms of percentages of the constituents, the powdered gelatin 110 makes up a range of about 55% to about 63% of the composition 100 by volume, the powdered flavoring 120 makes up a range of about 26% to about 34% of the composition 100 by volume, the powdered sweetener 130 makes up a range of about 5% to about 10% of the composition 100 by volume, and the powdered shelf stabilizer 140 makes up a range of about 2% to about 6% of the composition 100 by volume. In another embodiment where the powdered shelf stabilizer 140 makes up about 1 part of the composition 100 by volume, in terms of percentages of the constituents, the powdered gelatin 110 makes up a range of about 57% to about 61% of the composition 100 by volume, the powdered flavoring 120 makes up a range of about 28% to about 32% of the composition 100 by volume, the powdered sweetener 130 makes up a range of about 6% to about 9% of the composition 100 by volume, and the powdered shelf stabilizer 140 makes up a range of about 2% to about 6% of the composition 100 by volume. In a further embodiment where the powdered shelf stabilizer 140 makes up about 1 part of the composition 100 by volume, in terms of percentages of the constituents, the powdered gelatin 110 makes up about 59.3% of the composition 100 by volume, the powdered flavoring 120 makes up about 29.6% of the composition 100 by volume, the powdered sweetener 130 makes up about 7.4% of the composition 100 by volume, and the powdered shelf stabilizer 140 makes up about 3.7% of the composition 100 by volume.

In another embodiment, in terms of volumetric proportions, the powdered gelatin 110 makes up about 16 parts of the composition 100 by volume, the powdered flavoring 120 makes up about 8 parts of the composition 100 by volume, the powdered sweetener 130 makes up about 4 parts of the composition 100 by volume, and the powdered shelf stabilizer 140 makes up about 1 part of the composition 100 by volume. In an embodiment where the powdered sweetener 130 makes up about 4 parts of the composition 100 by volume, in terms of percentages of the constituents, the powdered gelatin 110 makes up a range of about 50% to about 60% of the composition 100 by volume, the powdered flavoring 120 makes up a range of about 25% to about 30% of the composition 100 by volume, the powdered sweetener 130 makes up a range of about 10% to about 15% of the composition 100 by volume, and the powdered shelf stabilizer 140 makes up a range of about 2% to about 6% of the composition 100 by volume. In another embodiment where the powdered sweetener 130 makes up about 4 parts of the composition 100 by volume, in terms of percentages of the constituents, the powdered gelatin 110 makes up a range of about 53% to about 57% of the composition 100 by volume, the powdered flavoring 120 makes up a range of about 26% to about 29% of the composition 100 by volume, the powdered sweetener 130 makes up a range of about 10% to about 15% of the composition 100 by volume, and the powdered shelf stabilizer 140 makes up a range of about 2% to about 6% of the composition 100 by volume. In a further embodiment where the powdered sweetener 130 makes up about 4 parts of the composition 100 by volume, in terms of percentages of the constituents, the powdered gelatin 110 makes up about 55.2% of the composition 100 by volume, the powdered flavoring 120 makes up about 27.6% of the composition 100 by volume, the powdered sweetener 130 makes up about 13.8% of the composition 100 by volume, and the powdered shelf stabilizer 140 makes up about 3.4% of the composition 100 by volume.

In another embodiment the powdered gelatin 110 makes up about 16 parts of the composition 100 by volume, the powdered flavoring 120 makes up about 8 parts of the composition 100 by volume, the powdered sweetener 130 makes up a range of about 2 parts to about 4 parts of the composition 100 by volume, and the powdered shelf stabilizer 140 makes up about 1 part of the composition 100 by volume. In an embodiment where the powdered sweetener 130 makes up a range of about 2 parts to about 4 parts of the composition 100 by volume, in terms of percentages of the constituents, the powdered gelatin 110 makes up a range of about 52% to about 62% of the composition 100 by volume, the powdered flavoring 120 makes up a range of about 25% to about 33% of the composition 100 by volume, the powdered sweetener 130 makes up a range of about 5% to about 16% of the composition 100 by volume, and the powdered shelf stabilizer 140 makes up a range of about 2% to about 6% of the composition 100 by volume. In another embodiment where the powdered sweetener 130 makes up about 2 parts to about 4 parts of the composition 100 by volume, in terms of percentages of the constituents, the powdered gelatin 110 makes up a range of about 53% to about 61% of the composition 100 by volume, the powdered flavoring 120 makes up a range of about 26% to about 32% of the composition 100 by volume, and the powdered sweetener 130 makes up a range of about 6% to about 15% of the composition 100 by volume, and the powdered shelf stabilizer 140 makes up a range of about 2% to about 6% of the composition 100 by volume.

In any of above described embodiments where the powdered sweetener 130 makes up about 2 parts of the composition 100 by volume, the powdered sweetener 130 comprises, for example without limitation, monk fruit sweetener. In any of the above described embodiments where the powdered sweetener 130 makes up about 4 parts of the composition 100 by volume, the powdered sweetener 130 comprises, for example without limitation, sugar. In any of the above-described embodiments where the powdered sweetener 130 makes up a range of about 2 parts to about 4 parts of the composition 100, the powdered sweetener 130 comprises, for example without limitation, one or both of monk fruit sweetener and sugar. In any of the above-described embodiments, the powdered flavoring 120 comprises powdered dried fruit, for example without limitation, powdered dried strawberries, powdered dried cherries, powdered dried blueberries, or other powdered dried berries. In any of the above-described embodiments, the shelf stabilizer 140 comprises, for example without limitation, arrowroot powder.

Without being held to theory, it has been found that any of the embodiments of the composition 100 described above, when applied under a person's upper lip, stops the person's nosebleed typically within one to two minutes of application. Delivery of the composition 100 does not require application of any particular pressure to the inside of the person's upper lip or gums. However, the application of the composition 100 can include being pushed up under the person's upper lip.

In this context the delivery of the composition 100 can be accomplished in multiple ways including, for example without limitation, by using a finger or an applicator. However, given that the composition 100 is being delivered into the person's mouth, it is undesirable for sanitary reasons to deliver the powder to under the person's upper lip using a finger. It is desirable and preferable that an applicator be used for delivery of the composition 100 to under the person's upper lip.

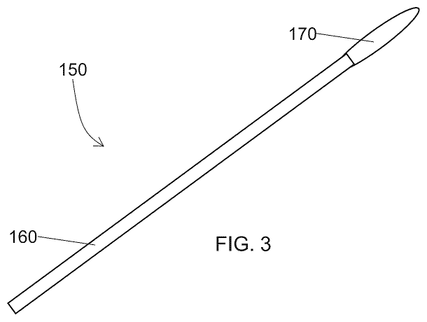

Referring to FIG. 3, an exemplary applicator 150 is illustrated having a body 160 and a tip 170 that is configured to pick up a powdered, gel, or cream material. For example without limitation, in an embodiment, the tip 170 is a brush, like a paint brush or the brush of a lip gloss applicator. In an embodiment the tip 170 is made of a material, for example without limitation, cotton, or a spongy material, to which the composition 100, comprising powdered components, would adhere when the material was wet. Either a brush type tip 170 or a spongy absorptive tip 170 would work well to pick up the composition in gel or cream form.

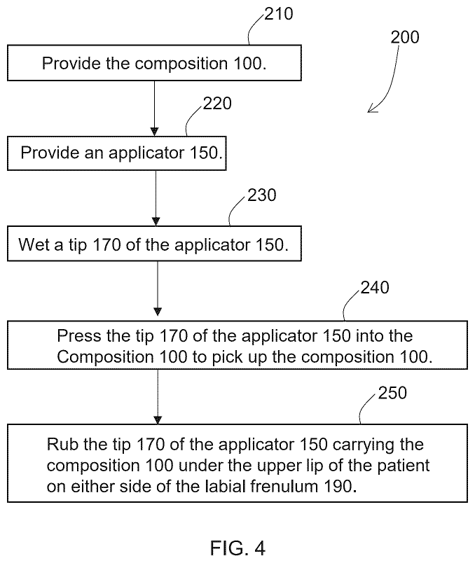

Referring now to FIG. 4 the steps in a method of treatment 200 for a nosebleed in a patient are shown. Starting at step 210, an embodiment of the above-described composition 100 is provided. At step 220, an embodiment of the applicator 150 is provided. If the composition 100 comprises components as noted above in a powdered form, it may be necessary to wet the tip 170 of the applicator 150 to pick up the composition 100. Therefore, at step 230, a tip 170 of the applicator 150 is wetted by a user. If the user is applying the treatment to themselves, the tip 170 of the applicator 150 may be wetted with the user's own saliva, for example, by licking the tip 170 of the applicator 150. However, if the user is applying the treatment to another person, it would not be desirable to wet the tip 170 of the applicator 150 with their own saliva. Rather, it is preferable for a user applying the treatment to another person to wet the tip 170 of the applicator 150 with water. In other embodiments, for example, if no water is available, the tip 170 of the applicator 150 can be wet with juice or milk or any liquid that would promote picking up the composition 100 comprising the components in a powdered form. In an embodiment the tip 170 of the applicator 150 can be wetted with the saliva of the person being treated. If the composition 100 comprises components as noted above in a gel or cream form, step 230 can be skipped.

At step 240, the tip 170 of the applicator 150, wetted as may be needed, is pressed into the composition 100 to pick up the composition 100. An effective amount of the composition 100 required to stop a nosebleed is typically that amount that can be carried on the tip 170 of the applicator 150. Without being held to theory, it has been found that when the wetted tip 170 of the applicator 150 is pressed into a quantity of the composition 100 comprising powdered components that about 1/12^{th} of a teaspoon or less of the powdered components get picked up by the wetted tip 170. This amount of the composition 100 is typically enough to stop a nosebleed in one to two minutes, as noted above. Therefore, an effective amount of the composition 100 comprising powdered components required to stop a nosebleed is about 1/12^{th} of a teaspoon or less.

However, in some cases a second application of the composition 100 may be necessary to stop the nosebleed. In cases where a second application is necessary, the effective amount of the composition 100 required to stop the nosebleed is twice the amount that can be carried on the tip 170 of the applicator 150, or about ⅙^{th} of a teaspoon or less. The effective amount of the composition 100 in gel or cream form will depend on how much the volume of the powder expands when packaged as a gel or a cream by the addition of a liquid. However, regardless of the volume of the gel or cream, the effective amount of the gel or the cream is that amount that contains about 1/12^{th} of a teaspoon or less of the composition 100 in powdered form, as noted above.

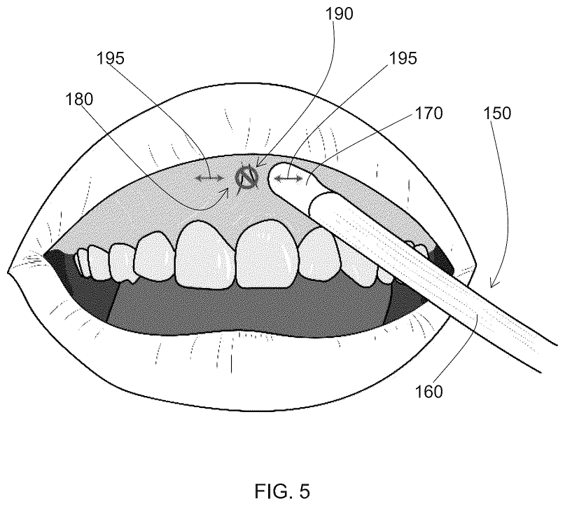

Referring now to FIGS. 4 and 5, at step 250, the tip 170 of the applicator 150 carrying the composition 100 is rubbed under the upper lip 180 of the patient on either side of the labial frenulum 190 where the upper lip of the patient meets the gums of the patient. In an embodiment, the tip 170 is rubbed generally horizontally as illustrated by arrows 195. In other embodiments the tip 170 can be rubbed vertically or in a combination of horizontal, vertical, and diagonal directions. As noted above, there is no particular pattern or quantity of pressure that needs to be applied by the tip 170 under the upper lip 180 of the patient. However, care should be taken to avoid the labial frenulum 190, which could block the motion of the tip 170 and/or cause pain to the patient.

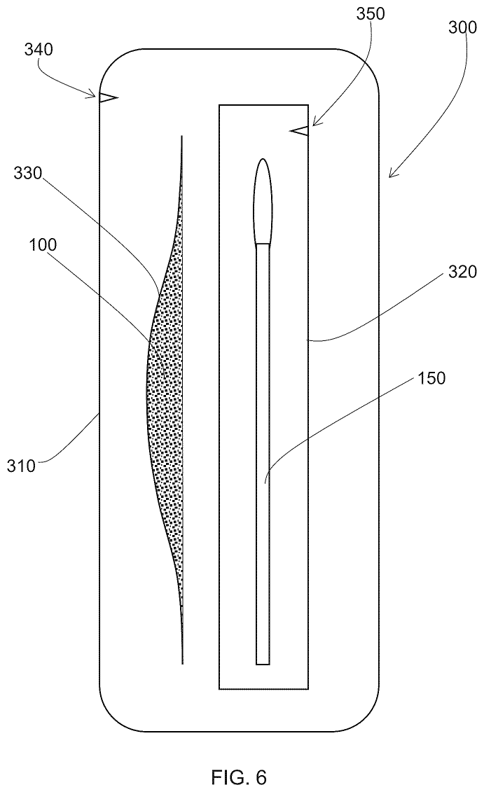

Referring now to FIG. 6, in an embodiment the items necessary for performing the method of treatment 200 are provided in an exemplary schematic drawing of an exemplary kit 300. In an embodiment the kit 300 is a single-use package 310 that contains an applicator 150 that is individually wrapped and sealed in a package 320 and the composition 100 that is sealed in another package 330. In an embodiment, the entirety of the composition 100 is sealed within a single package 330. In other embodiments, one or more of the constituents of the composition 100, whether in powder or gel or cream form is sealed in a separate package 330 such that there are two or more of the packages 330.

In another embodiment both the sealed package 320 and the one or more packages 330 share a common covering material and are backed by a common backing board or material and separated by a seal of the covering material against the common backing board. The seal can be oriented in any desired orientation relative to the sealed packages 320 and 330 so long as the applicator 150 and the composition 100 are sealed apart from one another. The seal can be via adhesive, sonic welding, or otherwise as appropriate to create the packages 320 and 330.

In an embodiment, the single-use package 310 can include two or more of the packages 320 each containing an applicator 10, or the package 320 can contain two or more of the applicator 150. In an embodiment the single-use package 310 includes a notch or line of weakness 340 that facilitates tearing the single-use package 310 open. Similarly, in an embodiment the package 320 containing the applicator 150 also includes a notch or line of weakness 350 that facilitates tearing the package 320 open. The packages 310, 320, and 330 may be made from plastic, or cardboard coated with plastic, or foil, or any material having any configuration as is known in the art for maintaining a sterile environment for the composition 100 and the applicator 150.

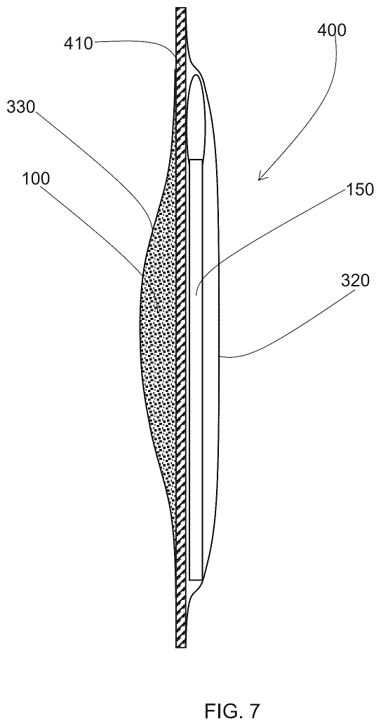

Referring to FIG. 7, in an embodiment, the packages 320 and 330 that contain the applicator 150 and the composition 100, respectively, can form a single-use package 400 by sharing a common sidewall 410 therebetween. In this embodiment, the composition 100 is deposited over a first side of the common sidewall 410 and sealed under material of an outer layer to form the package 330. The applicator 150 is then disposed on the other side of the common sidewall 410 and sealed under the material of the outer layer to form the package 320. The material forming the outer layers of the packages 320 and 330 can be the same material or different materials, and can be sealed via adhesive, sonic welding, or otherwise as appropriate to create the packages 320 and 330.

In an embodiment a plurality of kits 300 each comprising a single-use package 310, 400 as described above can be combined into a larger package. In an embodiment, each single-use package 310, 400 of the plurality of kits 300 can include two or more of the packages 320 each containing an applicator 150. In an embodiment, two or more of the packages 320 can be packaged together in another package (not shown) or two or more of the packages 330 can be packaged together in yet another package (not shown).

In the context of the kit 300 comprising the single-use package 310, an embodiment of the method of treatment 200 is described as follows. Steps 210 and 220 are executed by opening the single-use package 310, opening the package 320, and opening the package 330. Step 230 is executed as needed if the composition 100 comprises powdered components, by either licking the tip 170 of the applicator 150 (for self-application) or wetting the tip 170, for example, with water, juice, or milk (for self-application or for application to another). Step 240 is executed by pressing the tip 170 into the composition 100 to pick up a quantity of the composition 100 on the tip 170. Step 250 is executed, for example, as shown in FIG. 5 and as described above.

In an exemplary embodiment, the kit 300 includes the packages 320 and 330 within a single-use package 310, 400 for example, having any of the configurations as described above. In an embodiment the package 330 contains about ¼^{th} teaspoon of the composition 100 or less, which is about three times the effective amount of the composition 100 required to stop a nosebleed. In this embodiment a single-use package 310, 400 therefore contains enough of the composition 100 for three applications if necessary. In other embodiments, the package 330 contains more than about ¼^{th} teaspoon of the composition 100.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

What is claimed is:

1. A composition for treatment of nosebleeds, the composition comprising:
gelatin in a range of about 50% to about 67% by volume in powdered form;
flavoring in a range of about 23% to about 37% by volume in powdered form; and
sweetener in a range of about 5% to about 20% by volume in powdered form.

2. The composition of claim 1, comprising:
powdered gelatin in a range of about 50% to about 67% by volume;
powdered flavoring in a range of about 23% to about 37% by volume; and
powdered sweetener in a range of about 5% to about 20% by volume.

3. The composition of claim 2, comprising:
powdered gelatin in a range of about 54% to about 64% by volume;
powdered flavoring in a range of about 25% to about 33% by volume; and
powdered sweetener in a range of about 5% to about 18% by volume.

4. The composition of claim 2, wherein the powdered sweetener comprises one or both of sugar and monk fruit sweetener.

5. The composition of claim 2, wherein the powdered flavoring comprises powdered dried fruit.

6. The composition of claim 5, wherein the powdered dried fruit comprises powdered dried strawberries or powdered dried cherries.

7. The composition of claim 2, the composition further comprising powdered shelf stabilizer in a range of about 2% to about 6% by volume.

8. The composition of claim 7, wherein the powdered shelf stabilizer comprises arrowroot powder.

9. A kit, comprising:
a first package containing the composition of claim 1; and
a second package containing at least one applicator for application of the composition.

10. The kit of claim 9, further comprising an outer package containing the first package and the second package.

11. The kit of claim 9, wherein the second package contains two or more of the applicators.

12. The kit of claim 9, wherein the first package and the second package are sterile packages.

13. The kit of claim 12, wherein the first sterile package contains at least about 1/12^{th} of a teaspoon of the composition.

14. The composition of claim 1, wherein one or more of the gelatin, the flavoring, and the sweetener is in a gel or cream form.

15. A method of treatment for a nosebleed in a patient, the method comprising the steps of:
providing a composition, the composition comprising:
powdered gelatin in a range of about 50% to about 67% by volume;
powdered flavoring in a range of about 23% to about 37% by volume; and
powdered sweetener in a range of about 5% to about 20% by volume.; and
applying an effective amount of the composition under the patient's upper lip.

16. The method of claim 15, wherein the applying further comprises the steps of:
providing an applicator;
pressing the tip of the applicator into the composition to pick up the composition; and
distributing the composition under the upper lip of the patient on either side of the labial frenulum via the tip of the applicator.

17. The method of claim 16, wherein the distributing further comprises distributing the composition under the upper lip of the patient on either side of the labial frenulum via the tip of the applicator for a period of time between about a minute and about two minutes.

18. The method of claim 15, further including before pressing the tip of the applicator into the composition to pick up the composition, wetting the tip of the applicator.

19. The method of claim 15, wherein the effective amount of the composition is about 1/12^{th} of a teaspoon of the composition.

20. The method of claim 19, further including after the distributing, waiting about two minutes to determine if the nosebleed has stopped, and responsive to the nosebleed continuing:
pressing the tip of the applicator or a tip of a second applicator into the composition to pick up the composition; and
distributing the composition under the upper lip of the patient on either side of the labial frenulum via the tip of the applicator or the tip of the second applicator.
